(19) United States
(12) Patent Application Publication
(10) Pub. No.: US 2007/0154532 A1
(43) Pub. Date: Jul. 5, 2007

(54) Compositions comprising vitamins and/or derivatives thereof stablished with olea europea extract and/or ionene polymers

(75) Inventors: Matteo Tutino, Palermo (IT); Francesco Di Salvo, Palermo (IT)

Correspondence Address: CASELLA & HESPOS, 274 MADISON AVENUE, NEW YORK, NY, 10016, US

(73) Assignee: Med Care S.R.L., Palermo (IT)

(21) Appl. No.: 10/585,220

(22) PCT Filed: Dec. 24, 2004
(86) PCT No.: PCT/IT04/00727

§ 371 Date: Jun. 30, 2006

(30) Foreign Application Priority Data
Dec. 30, 2003 (IT) PA2003A000031
Jan. 19, 2004 (IT) PA2004A00001

(51) Int. Cl. A61K 36/28; A61F 13/02; A61K 31/785

(52) U.S. Cl. 424/448; 424/78.27; 424/769; 424/737

(57) ABSTRACT

The present invention relates to compositions comprising vitamins, such as L-ascorbic acid and its derivatives, sta-bilised with Olea Europea and/or ionene polymers. Particularly, the invention concerns stable compositions with high concentration of vitamins, for example L-ascorbic acid, to be advantageously employed in the medial and cosmetic field, for example for cosmetic and dermatological treatment of cute, mucous, and serosa.

The present invention relates to compositions comprising vitamins and/or derivatives thereof stabilised with Olea Europea extract and/or ionene polymers. Particularly, the invention concerns stable compositions with high vitamin concentration, such ascorbic acid, preferably L-ascorbic acid, and/or their derivatives, to be advantageously employed in the medical and cosmetic fields, for example for the cosmetic and dermatological treatment of skin and mucous membranes.

L-ascorbic acid, i.e. vitamin C, is widely employed in the medical field and particularly in the alimentary field, this substance being fundamental for the human organism. In fact, lack of this vitamin induces in human being a disease known as “scorbutus”. Being C vitamin fundamental for collagen, in the last decades it has raised a remarkable interest in the pharmaceutical cosmetic sector. Furthermore, it is known the application of C vitamin as antimicrobial and anti-oxidant additive for nutritional foods, or as purifying and disinfectant for pharmaceutical preparations such as cosmetics.

However, ascorbic acid in a serum solution has the tendency to the quick natural decomposition, even when it remains perfectly dissolved. L-ascorbic acid is readily soluble in water, but it quickly oxidises in aqueous solutions, and thus it cannot be stabilised with a sufficient concentration in this natural solvent. On the other hand, solubility of L-ascorbic acid in aqueous solutions is very limited. Instability of L-ascorbic acid in aqueous solutions is due to its alpha-ketonic structure, to its interaction with water, to unavoidable penetration of oxygen in the solution where vitamin has been dissolved, to the effect of the exposition to the light and to the time.

During the years different ways for preparing C vitamin (L-ascorbic acid) solutions stable for a reasonably long time have been suggested. Particularly, International Patent Application WO 98/23152 shows various preparation and action methods of C vitamin, both in its dextrogyrate form (ascorbyl palmitate) and in its levogyrate form (LLA), the latter being considered the form needed by the cell for activating its metabolism. Particularly, stabilisation of C vitamin is described solubilizing vitamin in a solvent, such as polyethylene glycol, ethoxydiglycol, propylene glycol, butylene glycol, propylene carbonate, glycerine, capric or caprylic glyceride, alkyl lactate, alkyl adipate, isosorbide, and their mixtures.

However, known methods do not allow obtaining L-ascorbic acid compositions with a sufficient intracellular penetration.

In view of the above, it is well evident the needing of having new alternative methods and compositions able to stabilise vitamins, particularly C vitamin, solving the drawbacks of the known compositions.

The applicant of the present invention has now found that some compounds usually employed separately in the pharmaceutical, dermatological and cosmetic formulations, such as Olea Europea, ionene polymers, some times in presence of polymeric ethers, tetraborates or thiosulfate, allow stabilising L-ascorbic acid in aqueous compositions. The above mentioned compounds allow preparing stable compositions with higher concentration of L-ascorbic acid in such a way of making said compositions more efficient, improving the intracellular, intranuclear, intra-mitochondrial penetration and optimising the bio-availability approaching the intracellular saturation. Furthermore, association between L-ascorbic acid and Olea Europea and/or ionene polymers extract develops a synergistic effect between the compounds of the composition thus able to increase the efficiency of each component. Aqueous extract of Olea Europea has been rarely used up to date as cosmetic topic treatment. This substance is a strong antioxidant and a microbicide, and, furthermore, by some peculiar substances, among which oleouropeina, exerts an important anti-tumorous action (Eur J. Cancer, 2000, Carcinogens. 2000). Efficiency of substances contained in the aqueous extract of Olea Europea is employed in natural medicine and for treatment of some dermatological and intestinal affections (Int J Antimicrob Agents, 2002). Said substance is some times used for exerting a regulation action of the immunitary system, also in case of allergy.

Ionene polymers are today employed in molecular biology as carriers. In fact, the facilitate the passage, by membrane proteins, of DNA and proteins (Bioconjug Chem 2002, J Control Release. 2002). These substances are thus used for the genic therapy, beside, in some particular form, as disinfectant products. Lack of cellular toxicity of ionene polymers is clearly demonstrated (Macromolecules, 1972). These substances can be employed as carriers and thus can replace in the cosmetic and pharmaceutical fields the use of liposome, nanosome, or other carriers presently used. At present, ionene polymers have never been used in combination wit Olea Europea or with ascorbic acid.

Ionene polymers are a large class of compounds, including also compounds having the general formula (I):
[—N(CH₃)₂—(CH₂)ₓ—N(CH₃)₂—(CH₂)_{y}-].2Z⁻ (I)
wherein x and y are integral numbers and Z is an halide. These polymers are obtained by reaction of N(CH₃)₂—(CH₂)ₓ—N(CH₃)₂ with Z-(CH₂)_{y}-Z (Macromolecules, 1972), for example ionene polymer obtained for reaction of 1,4-dichlorobutane with tetramethylethylendiamine.

Other ionene polymers are for example those obtained by reaction of 1,4-dichlorobutane with poly(oxyethylene(dimethylimino)ethylene(dimethylimino)ethylene dihalides), poly(2-hydroxyethylene-dimethyliminio-2-hydroxypropylene-dimethylimino methylene)dihalides, poly[{alkyl-(3-ammoniopropyl)imino}trimethylene dihalides], poly-[dialkyl-imino)ethylene halides] or with poly-[(hydroxy-dialkyl-imino)ethylene halides.

Olea and L-ascorbic acid (LAA) have important biological functions for skin and all the cellular tissues:
they have an antioxidant action opposing to “oxygen free” radicals stimulated by the same cellular metabolism and by the smoke, by the ultraviolet light exposition and by other polluting attacks, and they oppose to the cellular ageing;
have an anti-inflammatory action due to a strengthening of the immunitary system;
reinforce the cellular response to the outer and intracellular nociceptive stimuli;
exert a noticeable action in the modulation of the immunitary response;
intervene with an important antioxidant action in the body zones where an alteration of the cellular growth having a tumorous origin, a dysplastic alteration and/or a tissue atrophy is present;
stimulate synthesis of collagen in fibroblasts of human skin and particularly LAA intervenes in homeostasis of connective system of human organism. Furthermore, L-ascorbic acid acts for increasing synthesis of proteins and collagen, with anti-wrinkles effects, chelating action with respect to ferric ions, prevention of skin damages due to an excessive exposition to the sun (J Invest Dermatol. 1997, Radial Res. 2003).

L-ascorbic acid, Olea and ionene polymers play an important role in treatment and prevention of cancer, and this is due to the particular action of some substances contained in Olea Europea, such as oleuropeina and hydroxytyrosol, or ionene polymers that, combined with C vitamin, causing vitamin having a direct intracellular, intra-mitochondrion and citoplastic action (Cancer Immunol Immunother, 2003; Radiat Res, 2003).

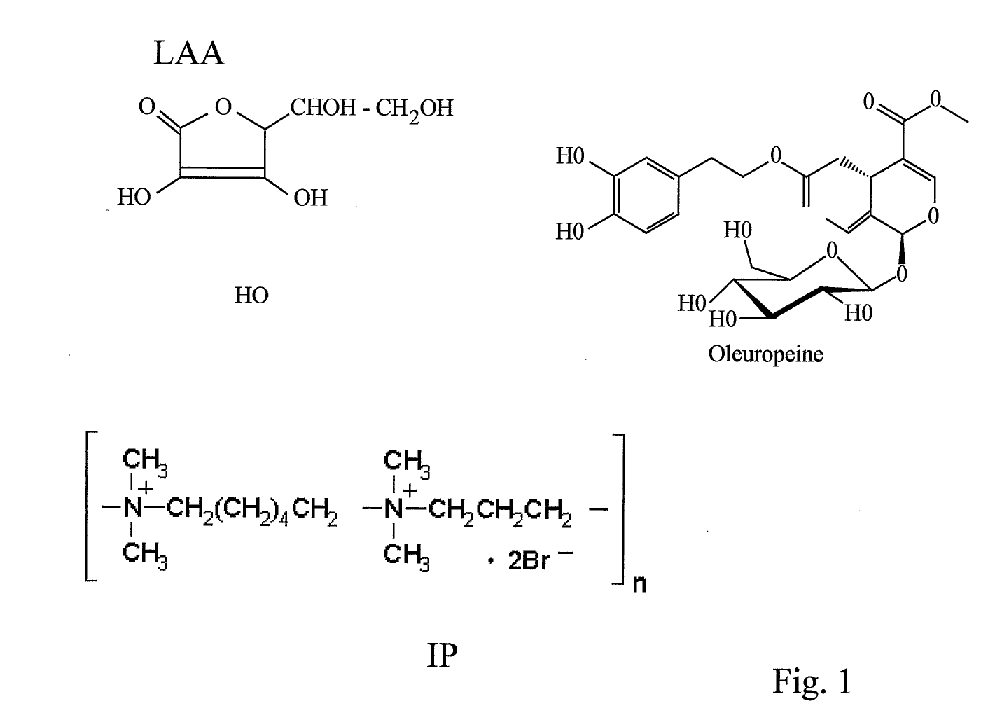

Olea Europea exerts an important action against hyperchromatism also by one of its components characterised by the presence of the benzene ring having an inhibition action again tyrosinase when applied topically. Further, aqueous extract of olives, containing oleuropeina, tyrosole and hydroxytirosole, increases the efficiency of C vitamin, conferring an extraordinary resistance against degradation. In fact, phenol group of oleuropeina contained in Olea Europea, being a replacement group, reacts with hydroxil group of C vitamin, eliminates water and creates a bond with the same preventing degradation and oxidation of L-ascorbic acid (FIG. 1). A solution of C vitamin has the possibility of oxidising and reversibly reducing from L-ascorbic acid into dehydrate L-ascorbic acid, thus phenol groups of oleuropeina react yielding two atoms of hydrogen to the dehydrate L-ascorbic acid restoring the L-ascorbic acid. Presence of a strongly reducing substance prevents the oxidation and thus the degradation process of C vitamin, conferring to the same a great stability.

By the term “Echinacea” different species of endemic plants from North America are indicated. Echinacea is from the Compositae family, and in the present classification of the Echinacea kind nine species and two varieties are indicated. However, only E. purpurea, E. angustifolia and E. pallida are used in therapy.

Results of many pharmacological studies have demonstrated that the various preparations that can be obtained by the aerial parts and by the roots of the medicinal plants of the Echinacea kind have the capability of stimulating the activity of the immunitary system, strengthening the functions of the natural killer cells and cyto-toxicity antibodies-dependent of the mononuclear cells of peripheral blood. Chemical components responsible of said pharmaceutical activity are a plurality: mainly polysaccharides (heteroxylanes and rhamno-arabino-galactanes), glycoproteins, cichoric acid, alkylamides, caffeic acid derivatives (echinacoside, Echinacea), terpenes, flavonoids (from leaves), rutin, caryophyllene. Further pharmaceutical effects of Echinacea are antiviral, bacteriostatic, fungistatic, anti-inflammatory, cicatrising activities.

It is therefore specific object of the present invention compositions comprising one or more vitamins and/or their derivatives in association with an Olea Europea extract, preferably an aqueous extract, and/or one or more of its components, such as oleuropeina, tyrosol and hydroxytyrosol, and/or one or more ionene polymers, along with one or more adjuvants and/or excipients pharmaceutically active or cosmetically acceptable.

Particularly advantageous are the compositions comprising ascorbic acid and/or its derivatives in association with an extract of Olea Europea, preferably an aqueous extract, and/or one or more of its components such as oleuropeina tyrosol and hydroxytirosol and/or one or more ionene polymers, along with one or more adjuvants and/or excipients pharmaceutically active or cosmetically acceptable.

Derivatives of ascorbic acid according to the present invention can be esters of ascorbic acid with fatty acids as, for example, ascorbyl palmitate and their salts.

Further vitamins, either hydro- and liposoluble vitamins, that can be present in the compositions according to the present invention are chosen in the group comprising vitamin A, B1, B2, B3, B5, B6, B8, B9, B12, D, E, K.

Compositions according to the invention can further comprise Echinea purpurea extract, angustifolia or pallida and/or one or more active principles contained therein, such as, for example, heteroxylanes, rhamno-arabino-galactanes, glycoproteines, cichoric acid, alkylamides, caffeic acid derivatives such as, for example, echinacoside and Echinacea, terpenes, flavonoids such as for example rutin, caryophyllene. In fact, extract of Echinacea or its active principles, besides having pharmaceutical activity, can further stabilise vitamins, particularly C vitamin.

Further, compositions can comprise one or more compounds chosen from the group comprising polymeric ethers, for example ethylene polymers or propylene oxides, monohydric alcohols, poly-hydric alcohols, for example etoxydiglycole, for example Transcutol®, also known as APV, tetraborates or thiosulfates.

In fact these compounds can be employed as adjuvant in the stabilisation of vitamins that, however is carried out in presence of Olea extract and/or ionene polymers.

According to a particular embodiment of the present invention, compositions can comprise one or more compounds chosen in the group comprising adenosine triphosfate, adenosine diphosfate, phosphoenolpyruvate, creatine phosphate, and inorganic phosphate. These compounds represent a source of cellular energy for generation of a proton gradient.

Above mentioned compositions can comprise also one or more substances included in the group comprising collagen, particularly type I collagen, fibrin glue, fibrin and its derivatives, dura mater. Association of ascorbic acid or its derivatives with one or more of the above substances is particularly advantageous in the treatment of wounds. In fact, these components are topic chemotactic agents of neutrophil, fibroblasts and/or endothelial cells. Above mentioned combination can be used in medical devices, such as gauzes, bandages, plasters, silicon bars, treated with the composition according to the invention or it can be employed for example as emulsion, gel, dust, paste, dispersion, solution. Wounds that can be treated with the compositions according to the invention are skin wounds, burns, sores, surgical ferrite, and chronic and acute lesions of skin and of mucous. Healing of wounds is accelerated and formation of anaesthetic cicatrix, such as keloids, hypertrophic cicatrix, is reduced, being promoted the production of I type collagen, and the cellular and extra cellular matrix regeneration processes are regulated along with all the relevant components.

Compositions according to the invention can further comprise one or more compounds chosen from the group comprising hyaluronic acid and/or its derivatives, for example esters, cross-linked or amidic derivatives, for example as gel, hydroxymethylcellulose, maltodextrines, fro example in injectable form, to be used as filler. Furthermore, compositions according to the invention can comprise one or more amino acids such as glycine, alanine, valine, leucine, isoleucine, serine, cysteine threonine, methionine, phenylalanine, tyroxine, tryptofano, histidine, lysine, arginine, aspartic acid, glutamic acid, asparagine, glutamine, proline.

Compositions according to the invention have a pH included in the range between 2.0 and 6.0 and, preferably, are in the serum, gel, emulsion and cream form.

As already mentioned in the above, said compositions can be used also for the preparation of gauzes, bandages, plasters, silicon bars, for example embedded with the compositions according to the invention. Therefore, a particular embodiment of the present invention is represented by medical devices comprising the compositions according to the invention.

Ionene polymers that can be employed have the general formula (I):
[—N(CH₃)₂—(CH₂)ₓ—N(CH₃)₂—(CH₂)_{y}-].2Z⁻ (I)
wherein x and y are integral numbers and Z is an halide, preferably Br or Cl. For example, one of the polymers can be obtained by reaction of 1,4-diclorobutano with tetramethylendiamine.

Other ionene polymers that can be employed are for example those obtained by reaction of 1,4-dichlorobutane with poly(oxyethylene(dimethylimino)-ethylene(dimethylimino)ethylene dihalides), poly(2-hydroxyethylene-dimethyliminio-2-hydroxypropylene-dimethylimino methylene)dihalides, poly[{alkyl-(3-ammoniopropyl)imino}trimethylene dihalides], poly-[dialkyl-imino)ethylene halides] or with poly-[(hydroxy-dialkyl-imino)ethylene halides.

Composition according to the invention can comprise ascorbic acid or its derivatives at a concentration between 0.1% and 35%, preferably between 10% and 25%, still more preferably between 12% and 20%. Vitamin A (trans and cis-retinoic acid) can be present at a concentration between 0.001 % and 10%, preferably between 1.5% and 3.2%. Extract of Olea Europea and/or one or more of its components, such as oleuropeina, tyrosol and hydroxytyrosol can be present at a concentration between 0.1% and 95%, preferably between 5% and 50%, still more preferably between 15% and 30%. Polymeric ethers can be present at a concentration between 5% and 50%, mono and/or poly-hydric alcohols at a concentration between 5% and 50%, thiosulfates at a concentration between 0% and 5%, preferably between 0.01 % and 3%.

Echinacea extract and/or one or more of its active principles can be present at a concentration between 0.001% and 10%, as well as one or more compounds of the group comprising adenosine triphosfate, adenosine diphosphate, phosphoenolpyruvate, creatine phosphate, inorganic phosphate can be present at a concentration between 0.001% and 35%.

The above mentioned percentages are weight percentages of the components with respect to the weight of the composition.

Particularly, thiosulfates are provided as potassium and/or sodium and/or ammonium salts.

Thus, they are compositions comprising high concentrations of vitamins, preferably L-ascorbic acid, stabilised along with other components improving their efficiency, intracellular, intranuclear, intramitocondrial penetration, resistance to the oxidation, hydration capability, use pleasure.

A specific example of the composition according to the invention can be: 20% water, preferably bi-distilled water, 20% L-ascorbic acid, 2.66% ionene polymer having formula (I), with x=3 and y=3, 0.3% potassium thiosulfate, 15% aqueous extract of Olea Europea, 42% of a mixture 50:50 of propylenglycol and 1,2 butylenglycol.

A further example is represented by: 20% water, preferably bi-distilled water, 20% L-ascorbic acid, 2.66% ionene polymer having formula (I), with x=3 and y=3, 0.3% potassium thiosulfate, 22% aqueous extract of Olea Europea, 15% polioxyethylene/polioxypropylene polymer, 20% of a mixture 50:50 of propylenglycol and 1,2 butylenglycol.

A further specific example of composition according to the invention is represented by: 14.2% L-ascorbic acid, 26.8% aqueous extract of Olea Europea, 44% etoxyglycol, 0.06% citric acid, 14.2% depurated water.

Compositions object of the present invention can contain disinfectant additives, bactericides and DNA correctors having an intranuclear, mitochondria and cytoplasmatic action such as oleuropeina, tyrosol and hydroxytyrosol, enzymes and catalysers of the intracellular biochemical energetic reactions. As to the use in the medical field of ionene polymers, data relevant to pharmacological and clinical tests have been widely published in international magazines.

Olea Europea, for example as aqueous extract, and ionene polymers, beside a disinfecting and bactericide function, have at the same time a strong stabilising action with respect to vitamins, particularly of L-ascorbic acid in solution. These compounds further promote penetration through membrane proteins.

A particular embodiment of the present invention concerns compositions comprising vitamin A and E in association with one or more ionene polymers, along with one or more adjuvants and/or excipients, pharmaceutically or cosmetically acceptable. This composition is particularly suitable for treatment of skin, mucous and serosa. It is able to promote the biosynthesis of coliagen, repairing cellular damages, stabilising and strengthening the action of the substances employed in the medical, dermatological and cosmetic field, promoting a perfect penetration of the complexed substances. The composition can penetrate from the extra-cellular space inside the cell and exerting the intracytoplasmatic action also acting on the biosynthesis of proteins and, at the same time, having an action within the cellular nucleus stabilising DNA, increasing the resistance to the nociceptive stimuli and, in case DNA is already damaged, promoting a quick reparation of the same.

Vitamin A (trans and cis-retinoic acid) can be present at a concentration between 0.01% and 5%. Also vitamin E (alpha tocopherol), for example in its acetate form, can be present at a concentration between 0.01% and 5%. Ionene polymers can be present at a concentration between 0.001% and 10%, preferably between 1.5% and 3.2%.

The above mentioned composition can further comprise one or more compounds chosen in the group comprising cogic acid, azelaic acid, fitic acid, glycolic acid, lactic acid, fumaric acid, tartaric acid, L-aspartic acid, L-ascorbic acid, Phytic acid, Arbutine. The above mentioned compounds can be present at a concentration between 1.5% and 20%.

Particularly, the composition can further comprise one or more emollients, such as cetyl esters, glycerine, flowing such as stearyl alcohol, cheryl alcohol, emulsifying agents such as cetyl alcohol, glycerine, sodium lauryl sulfate, preservatives such as methylparaben, surface-active such as Quaternium/15, fungicides such as propylparaben.

Cetyl esters having a synthetic origin and in any case not distinguishable from waxes derived from natural spermaceti as far as chemical composition and properties are concerned. These esters are comprised of a mixture of esters of fatty acids containing between 14 and 18 atoms of Carbon along with alcohols and they can be included in the formulations as emollients or “softening agents”. Cetyl esters can be present in the formulation at a concentration between 0.1% and 10%, preferably between 5% and 9%. Stearyl alcohol can be present at a concentration between 0.1% and 15%, preferably between 5% and 12%, cheryl alcohol between 5% and 12%, cetyl alcohol between 0.1% and 6%, preferably between 2% and 6%, glycerine between 0.1% and 18%, preferably between 2 and 12%, laurylsulfate sodium between 0.1% and 1.5%, preferably between 0.5% and 1.0%.

Cetyl ester, stearyl alcohol, cheryl alcohol, and glycerine form a hydrating basic cream promoting the application on the skin with positive effects, preservation of the composition is further promoted by the presence of methyl paraben between 0.1 % and 0.4%, preferably between 0.05% and 0.3%, propyl paraben between 0.01% and 0.1%, preferably between 0.02% and 0.05%, surface-active Quaternium/15 between 0.01% and 0.15%, preferably between 0.05% and 0.12% deionised or distilled water is present in the formulations according to the present invention as inert carrier acting as diluent and at the same time has wetting properties.

According to a particular embodiment of the present invention, the composition comprises: ionene polymer between 0.01% and 10% in weight, cogic acid between 1% and 10% in weight, azelaic acid between 1 % and 30% in weight, glycolic and/or lactic acid between 1.5% and 15% in weight, trans-cis retinic acid between 0.01% and 5% in weight, acetate E vitamin between 0.5% and 5% in weight, hydrating base cream comprising at least an emollient or wetting-agent selected from a group comprising cetyl esters, cetyl alcohol, glycerine, a preservative selected from the group comprising methyl paraben, propyl paraben and laurylsulfate sodium as emulsifying agent, and finally water up to 100% in weight.

Preferably, the above mentioned compositions are in cream form. It is further object of the present invention the use of the compositions according to the invention for the cosmetic treatment, for example for treatment of wrinkles, of skin spots.

Compositions according to the present invention can be further advantageously used in the medical field, both the treatment of the humans and in the veterinary field.

Particularly, it is object of the present invention the use of the compositions according to the invention for the preparation of a medicament for treatment of keratosis actinic, of wounds, of sores, of diabetic cutaneous sores, of lesions of oral mucous, of psoriasis, for prevention and treatment of skin tumours in general, and of the acute and chronic lesions of skin.

Compositions according to the present invention can be used on the human body, particularly on the skin, in association with pulsed light laser technology and particularly within wavelengths between 520 and 670 nm, and more particularly 650 nm, determining a proton gradient according to the Andrè Jagendorf law.

Furthermore, the present invention concerns the use of ionene polymers and/or Olea Europea extract for stabilising compositions of vitamins or their hydro- or lipo-soluble derivatives, fro example in form of aqueous solutions or emulsions, particularly compositions comprising one or more vitamins, or their derivatives, chosen from the group comprising vitamin A, B1, B2, B3, B5, B6, B8, B9, B12, D, E, K, C.

Compositions according to the present invention can be prepared according to the preparation techniques well known to those skilled in the art.

The present invention will be now described, for illustrative but not limitative purposes, according to its preferred embodiments, with particular reference to the figures of the enclosed drawings and examples, wherein:

FIG. 1 shows a scheme of interaction between L-ascorbic acid and oleuropeina, one component of Olea Europea or ionene polymer;

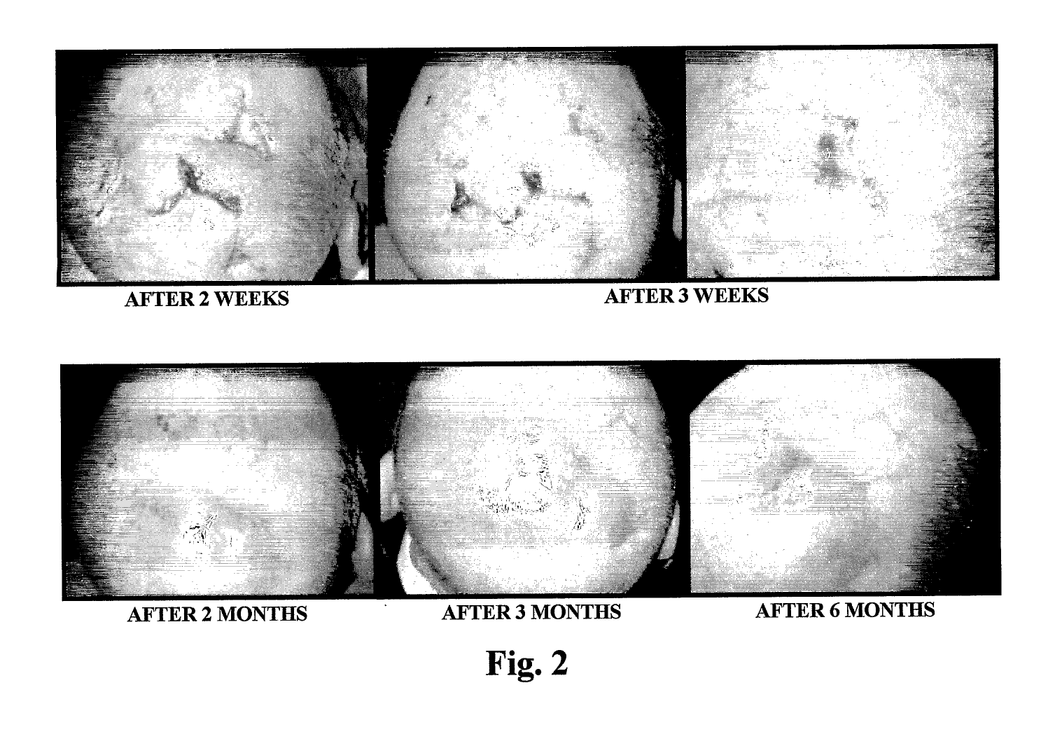

FIG. 2 shows the results of case 1, example 5;

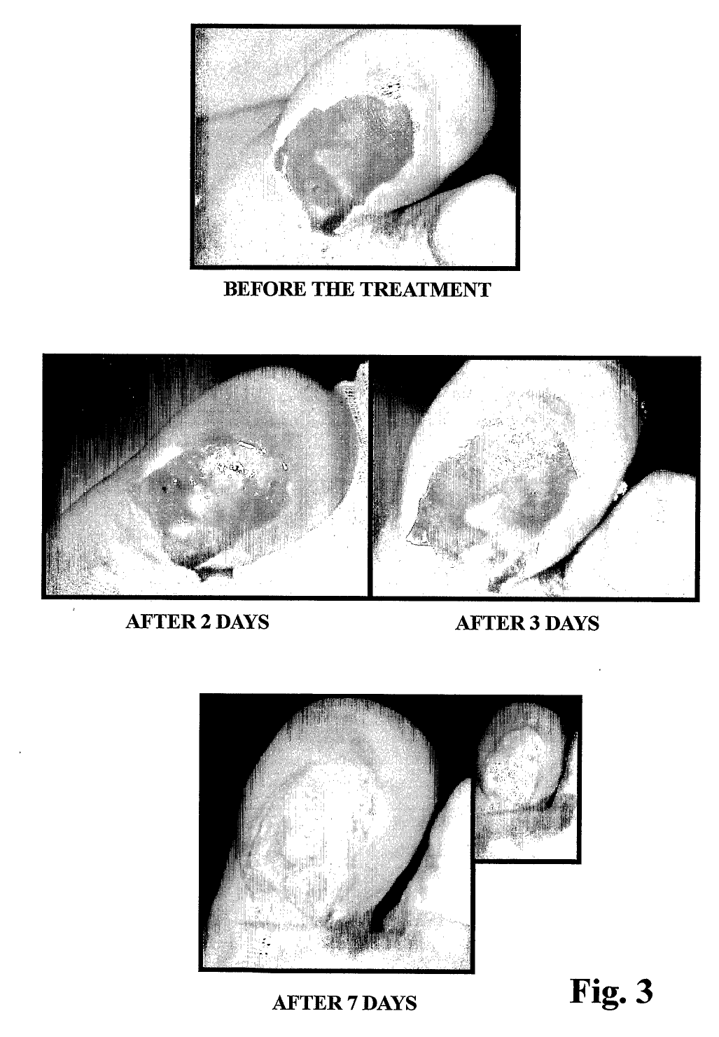

FIG. 3 shows the results of case 2, example 5;

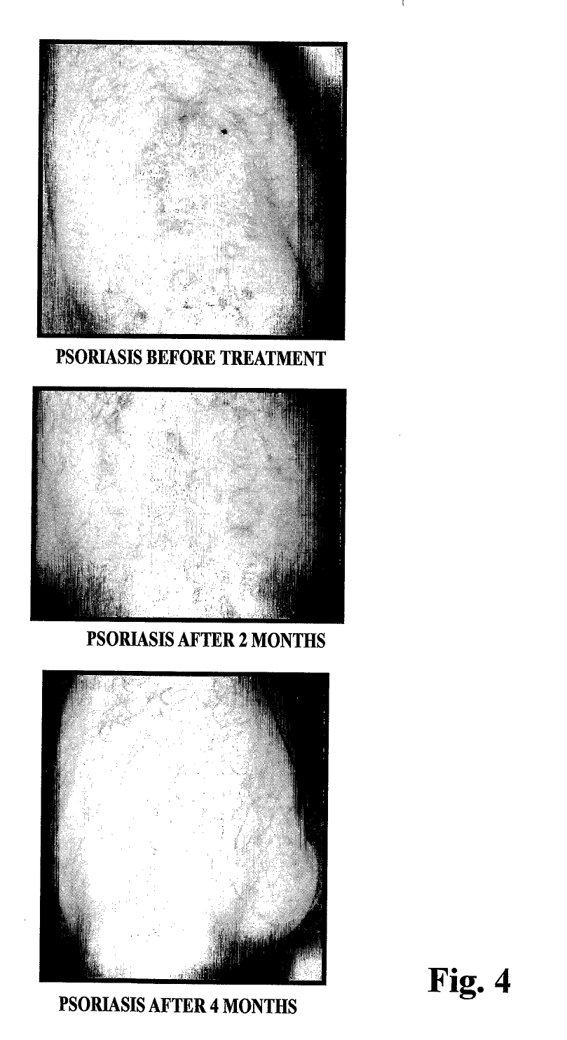

FIG. 4 shows the results of case 9 (elbow psoriasis), example 5;

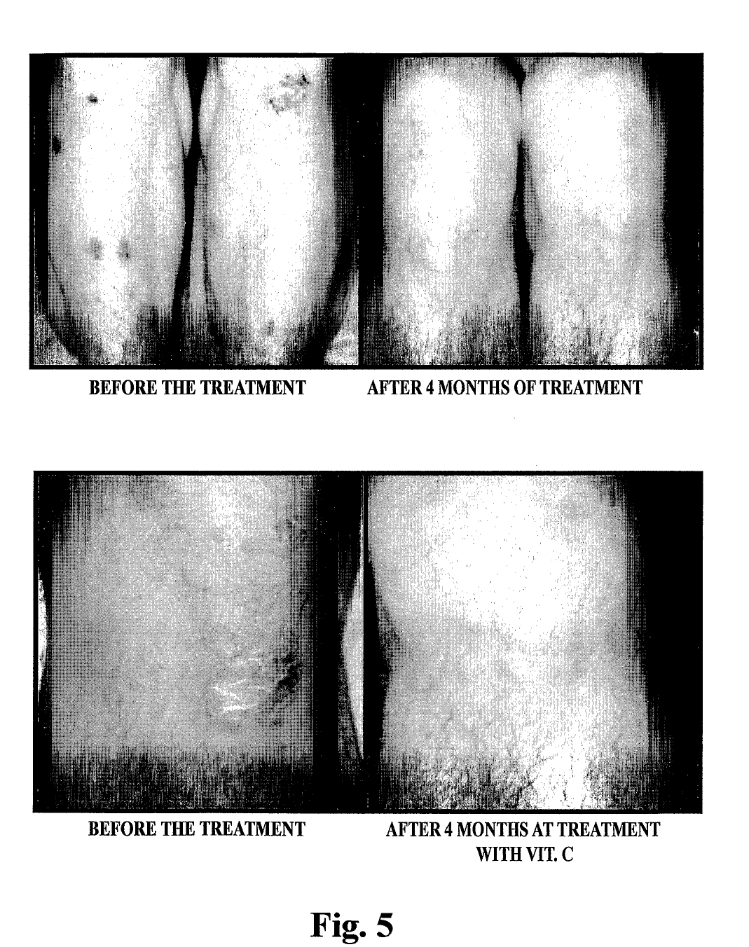

FIG. 5 shows the results of case 9 (leg psoriasis), example 5;

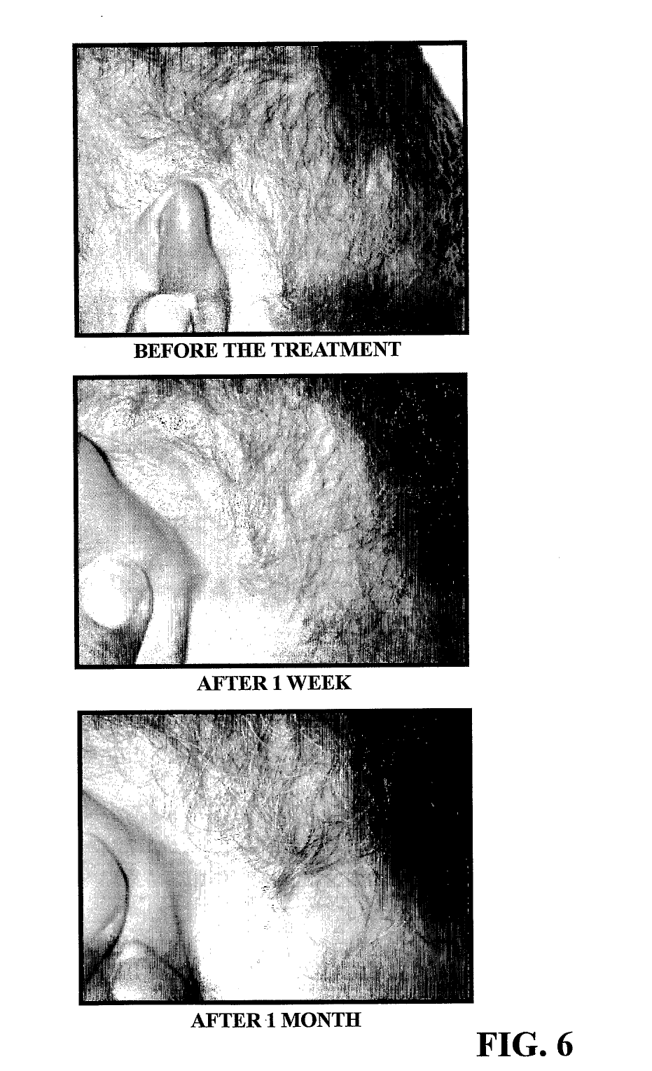

FIG. 6 shows the results of case 9 (mastoid retroauricolar region psoriasis), example 5.

### EXAMPLE 1

### Study of Stability of a Composition According to the Invention with Respect to a Comparative Formulation

The following concentrations are given in weight %:
20% L-ascorbic acid
20% bi-distilled water
2.66% ionene polymer of general formula (I) (with x=3 and y=3).
0.3% potassium thiosulfate
15% SYNPERONIC P94 (polyoxyethylene/polyoxypropylene polymer)
22% aqueous extract of Olea Europea
20% of a 50:50 mixture of propylene glycol and 1,2 butylene glycol.

In this formulation, active package stabilising the L-ascorbic acid is comprised of the following compounds: aqueous extract of Olea Europea, ionene polymer, polymer and copolymers of ethylene glycol and butylene glycol, polymer comprised of the copolymerisation of polyoxyethylene-polyoxypropylene and thiosulfates.

This solution is addressed to the pharmaceutical, dermatological, cosmetic fields, as well as to the medical-surgical and veterinary fields, and to the treatment of wounds and sores of biological tissue, and more specifically of skin.

In the latter case, for example, equine collagen, or other kind of collagen, can be impregnated with the same solution according to the present invention, and according to method already known to those skilled in the art.

It has been prepared the following comparative solution (weight percentages)
20% bi-distilled water
20% L-ascorbic acid

50% of a solution at 50% of polioli.

COMPARATIVE TEST
Composition according to the invention

| | |
|---|---|
| After 0 months | After 25 months |
| Slightly yellow colour | unchanged colour |
| Degradation 0% | Degradation 5%-9.8% |

Composition according to the comparative formula

| | |
|---|---|
| After 0 months | After 25 months |
| White colour | dark brown colour |
| Degradation 0% | Degradation 98% |

### EXAMPLE 2

### Study of Stability of a Composition According to the Invention with Respect to a Comparative Formulation

The following concentrations are given in weight %:
20% L-ascorbic acid
20% bi-distilled water
2.66% ionene polymer of general formula (I) (with x=3 and y=3)
0.3% potassium thiosulfate
15% SYNPERONIC P94 (polyoxyethylene/polyoxypropylene polymer)
42% of a 50% mixture of propylene glycol and 1,2 butylene glycol.

In this formulation, active package stabilising the L-ascorbic acid is comprised of the following compounds: ionene polymer, polymer comprised of the copolymerisation of polyoxyethylene/polyoxypropylene and thiosulfates.

This solution is addressed to the pharmaceutical, dermatological, cosmetic fields, as well as to the medical-surgical and veterinary fields, and to the treatment of wounds and sores of biological tissue, and more specifically of skin.

In the latter case, for example, equine collagen, or other kind of collagen, can be impregnated with the same solution according to the present invention, and according to method already known to those skilled in the art.

It has been prepared the following comparative solution (weight percentages)
20% bi-distilled water
20% L-ascorbic acid

50% of a solution at 50% of polioli.

COMPARATIVE TEST
Composition according to the invention

| | |
|---|---|
| After 0 months | After 25 months |
| Slightly yellow colour | Slightly yellow colour |
| Degradation 0% | Degradation 9.5% |

Composition according to the comparative formula

| | |
|---|---|
| After 0 months | After 25 months |
| White colour | dark brown colour |
| Degradation 0% | Degradation 98% |

### EXAMPLE 3

### Composition of L-ascorbic Acid at 14.2% Stabilised with Olea Europea Extract and Ionene Polymers

| | |
|---|---|
| L-Ascorbic acid | 14.2% |
| Ionene polymer POLIBREN ® | 0.34% |
| Aqueous extract of Olea Europea | 26.5% |
| Etoxydiglycol | 44.0% |
| Citric acid | 0.06% |
| Depurated water | 14.2% |

Slightly yellow composition remains unchanged after 25 months.

### EXAMPLE 4

### Composition of L-ascorbic Acid at 14.2% Stabilised with Olea Europea Extract and Ionene Polymers

| | |
|---|---|
| L-Ascorbic acid | 14.2% |
| Aqueous extract of Olea Europea | 26.8% |
| Etoxydiglycol | 44.0% |
| Citric acid | 0.06% |
| Depurated water | 14.2% |

Slightly yellow composition remains unchanged after 25 months.

### EXAMPLE 5

### Clinical Studies on Results of the Application of Composition According to Example 3 and/or 4

Different clinical applications of the compositions according to the present invention have been carried out, said compositions being based on LAA stabilised by aqueous extract of Olea Europea and creams containing vitamin E, A and its derivatives, all complexed with Olea, including cogic or azelaic acid. Practically, clinical application was carried out on 50 subjects affected by various cutaneous alterations, such as: bedsores, diabetic sores, various cases of cutaneous tumours from dysplasia to actinic keratosis (pre-cancerous), thermal and chemical burns, cutaneous ageing, surgical cicatrix and other kinds of cicatrix, different kind of acne, cutaneous atrophy, cutaneous dehydration, psoriasis and other dermatological pathologies, with interesting results; therefore, some random cases are listed in the following among the 50 cases subjected to treatment.

Exemplificative case no 1: 74 year-old patient affected by basocell carcinoma recurrence of scalp. Patient is subjected to surgical intervention, tumour is removed and area is covered by transposition multiple flaps for the whole thickness. In seats where it was not possible realising a precise cutaneous approaching of the cutaneous edges, and thus uncovered areas was present with exposition of a bone of cyanic theca, sheets of equine collagen are placed. Then, daily medications are made on the part subjected to intervention employing LAA 14.2% complexed with Olea and/or ionene in serum form. Medications are carried out also in the area comprising the whole scalp, since affected by pre-cancerous such as actinic keratosis, tumorous lesions and cutaneous atrophy zones. After two weeks, it is noted a remarkable reepithelialization at the edges of the surface surgical wounds to the collagen, undoubtedly working as bridge for the cellular structures that, from the edges, reepitheliase the whole surface. Healing of the wound is completed within 3 weeks from the intervention. Prosecuting the treatment with stabilised LAA and vitamin A based creams and its derivatives, complexed with Olea, the perfect restoring of the whole surface affected by actinic keratosis and epithelioma. Cutaneous surface treated after three months has assumed the normal aspect (FIG. 2).

Exemplificative case no 2: A 42 years old patient with uncompensated diabetes (values 340 mg/dl) affected by diabetic sore on the first finger of the left foot, where also paresthesias are present. It is daily medicated with LAA complexed with Olea and/or ionene and fatty gauzes. After seven days, the complete reepithelialization and restoring of sensitivity is obtained (FIG. 3).

Exemplificative case no 3: this case concerns the application for cosmetic use of vitamin C (LAA) complexed with Olea and/or ionene and creams with vitamin A and vitamin E, complexed with Olea and/ionene. A 26 year old patient, having solar spots on neck and “lentigo solaris”. Serum object of the present invention is applied with creams containing as active principle, substances such as derivatives of vitamin A, C and E, complexed with Olea. Whitening action is mainly obtained by serum of vitamin C and partially also by creams containing cogic acid or azelaic acid stabilised with Olea. After 8 weeks from the beginning of the treatment, patient is happy, lentigo solaris completely disappeared, cute is soft, well hydrated and with a uniform colour.

Exemplificative case no 4: this case concerns the application for cosmetic use of vitamin C complexed with Olea in combination with creams containing vitamin A, C and derivatives, vitamin E and cogic and/or azelaic acid, complexed with Olea. A 48 year old patient, suffering of acne rosacea and diffuses teleangectasys on the face, also due to the previous treatment with Retin A. patient has been treated with creams comprised of the above active substances, complexed with Olea, Salicylic and malaleuca alternifolia and vitamin C, as serum. After a treatment lasting 4 weeks, patient was subjected to a chemical peeling with 20% TCA and it was created a chemical caustic up to the basal layer of epidermis, reaching on some points the IRD (immediate reticular derma). Patient was subjected to a domiciliary treatment with creams and reepithelializing substances based on AA, Aloe, Vitamin C, A, E complexed with Olea and/or ionene. At the beginning of the exfoliation phase on new skin, vitamin C has been dispersed on new skin. Patient reached the complete reepithelialization within and not beyond 7 days, obtaining extraordinary results. After exfoliation, patient started again with treatment based on creams at very low dosages to maintain the result. It is to be noted that after exfoliation, skin had not a red colour.

Exemplificative case no 5: A 20 year old patient with fatty skin, hyperchromatism and hirsutism. At the basis of hormonal alteration concerning testosterone. Treatment is started with creams, astringent and vitamin C stabilised in form of serum. After two months of treatment a uniform colour has been obtained, as well as reduction of fat secretion and reduction of pores dimension.

Exemplificative case no 6: A 72 year old patient with wrinkles and noticeable ageing. A treatment starts for 3 months using the composition according to the invention, intercalate with a peeling with TCA 20%. An evident improvement of wrinkles and whitening action of substances used for restoring the colour and the cutaneous tonicity.

Exemplificative case no 7: A 44 year old patient with cutaneous spots, photo-ageing. Keratosis actinic and lost of cutaneous elasticity. A treatment starts with creams according to the present invention applied twice a day. It is controlled every two weeks, adjusting the dosage according to the results of the patient to the therapy with creams and serum according the present invention. Patient is completely cutaneous spots free after 8 weeks of daily treatment and a TCA 20% peeling up to the plane corresponding to the basal membrane.

Exemplificative case no 8: A 45 year old patient subjected to intervention for foot sarcoma, to which a free edge of gran dorsale was carried out. Patient was subjected after the intervention to radiant oncology therapy, causing dystrophic sores due to the radiotherapy. Patient has treated the irradiated part with the serum according to the present invention, twice a day, and after a period of 7 days, not only sores were completely reepithelialized, but also even more an important anti-inflammatory action of the part subjected to treatment was noted.

Exemplificative case no 9: A 48 year old patient suffering of psoriasis guttata for 20 years, tried different therapies without positive results. After three months of treatment using the composition according to the invention, healing with disappearing of psoriasis lesions localised on the head and at the articulation level (FIG. 4-6) was reached.

The above cases have been randomly chosen among the 50 cases subjected to treatment. Action of LAA complexed with ionene polymers and/or Olea Europea, not only in serum form but also as creams, comprising vitamin C, vitamin A and their derivatives, vitamin E, ascorbic acid and azelaic acid, Arbutin and Fitic acid, all complexed with ionene polymers and/or Olea Europea. Particularly, action of Olea Europea and/or of ionene polymers when complexed, is synergic in efficiency and action for:
prevention and treatment of spots (preventive action when exposed to sun rays, and in the post-inflammatory pigmentation, PIH);
treatment and prevention of skin tumours, lentigo senilis, lentigo solaris, etc.;
treatment of actinic keratosis;
treatment of surgical wounds since the whole participates in the healing processes;
restoring of cutaneous functionality and homeostasis;
restoring and stimulation of normal cellular hydration;
restoring of regular cutaneous elasticity and of functions relevant to collagen action;
treatment of bedsores, diabetic cutaneous sores and so on;
treatment of oral mucous lesions;
cutaneous and mucous reepithelialization processes;
treatment of Acne;
treatment of burn sores;
treatment of surgical wounds also when combined with use of collagen where collagen exerts also a support action to the process of reepithelialization stimulated by vitamin C complexed with ionene polymer.
treatment of surgical accidents in intestinal surgery when it is wished preventing the beginning of cicatricial bridles, since said substances are modulators of the collagen synthesis;
treatment of skin diseases in cases when it is possible providing the use of substances containing vitamin C, A, E, ecc. complexed with ionene polymers;
treatment of cicatricial and acute consequences of histic infarct, also cardiac infarct, being the substances important mediators for healing;
treatment of oral mucous lesions, including aphtas and herpes;
treatment of cutaneous herpes.

Compositions according to the present invention exert their action both on the nervous system, stimulating a repair of nerves both on the epithelial tissues where surely complex with vitamin C plays a key role particularly for creation of collagen.

It has been demonstrated that both vitamin C and Olea Europea have an action in preventing skin tumours and particularly exerts an action protecting from damages of DNA caused by UVB (Cancer Immunol lmmunother.2003 November;52—Eur. J. Cancer. 2000 June;36 (10):1235/47).

Furthermore, it has been noticeably noted that complex of Olea, Vitamin C (LAA), ionene and vitamin A and derivated complexed with ionene and/or Olea Europea) inhibit and reduce collateral effects due to the use only of vitamin A and derivatives, such as new-production of vessels, cutaneous teleangectasys surely inducing unaesthetism. Said protection of the membrane vessel is due to the action of complex vita C—ionene polymers or vitamin C oleuropeina and this clinically noted on patients having a cute with a perfect vascularisation, hydration, where presence of telengactasys is irrelevant.

Considering the action of vascular protection of complex LAA—Olea Europea—and/or ionene, the latter has its maximum therapeutically result in association with tretinoine complexed with ionene and/or Olea when treating rosacea, i.e. a case with a rich vascular component.

It is further to be noted that complex LAA—Olea Europea and/or ionene polymer accelerates healing processes on skin exfoliated following to a peeling.

### EXAMPLE 6

### Composition Comprising Iionene, Vitamin A and Vitamin E According to the Invention

The following concentrations are given in weight %:

| | |
|---|---|
| Ionene polymer | 2.1 |
| Cis retinic acid (vitamin A) | 0.08 |
| Vitamin E acetate | 0.5 |
| Cogic acid | 4.2 |
| Cetyl Ester | 8.4 |
| Cetyl alcohol | 4.0 |
| Glycerine | 10 |
| Methyl paraben | 0.2 |
| Propyl paraben | 0.02 |
| Cationic surface-active Quaternium/15 | 0.1 |
| Laurylsulfate Sodium | 2.5 |
| Deionised water | Up to 100% |

### EXAMPLE 7

### Composition Comprising Ionene, Vitamin A and Vitamin E According to the Invention

The following concentrations are given in weight %:

| | |
|---|---|
| Ionene polymer | 2.1 |
| Cis retinic acid (vitamin A) | 0.05 |
| Vitamin E acetate | 0.5 |
| Cogic acid | 4.2 |
| Azelaic acid | 20 |
| Glycolic acid | 2.1 |
| Cetyl ester | 8.4 |
| Glycerine | 10 |
| Methyl paraben | 0.2 |
| Propyl paraben | 0.02 |
| Cationic surface-active Quaternium/15 | 0.1 |
| Laurylsulfate Sodium | 2.5 |
| Deionised water | Up to 100% |

### Bibliography

WO 98/23152
Eur J. Cancer. 2000 June; 36 (10): 1235-47 The antioxidant/anticancer potential of phenolic compounds isolated from olive oil. Owen R W, Giacosa A, Hull W E, Haubner R, Spiegelhalder B, Bartsch H.
Carciogenesis. 2000 November; 21 (11): 2085-90. Protective effect of topically applied olive oil against photocarcinogenesis following UVB exposure of mice. Budiyanto A, Ahmed N U, Wu A, Bito T, Nikaido O, Osawa T, Ueda M, Ichihashi M.
J Control Release. 2002 May 17:81(1-2):201-17. Physical properties and in vitro transfection efficiency of gene delivery vectors based on complexes of DNA with synthetic polycations. Reschel T, Konak C, Oupicky d, Seymour L W, Ulbrich K.
Cancer Immunol Immunother. 2003 November; 52 (11) 693-8. Epub 2003 Jun. 24 L-ascorbic acid (vitamin C) induces the apoptosis of B16 murine melanoma cells via a caspase—8—independent pathway. Kang J S, et all.
J Invest Dermatol. 1997 March; 108 (3): 302-6. L-ascorbic acid inhibits UVA-induced lipid peroxidation and secretion of IL-1alpha and IL-6 in cultured human keratinocytes in vitro. Tebbe B. et all.
Bioconjug Chem 2002 May-June; 13(3):548-53. Aliphatic ionenes as gene delivery agents: elucidation of structure-function relantionship through modification of charge density and polymer length. Zelikin An, Putnam D, Shastri P, Langer R, Izumrudov V A.
Int J Antimicrob Agents 2002 October;20(4):293-6. In vitro antimycoplasmal activity of oleuropein. Furneri P M, Marino A, Saija A, Uccella N, Bisignano G.
Macromolecules, Vol. 5 page 253. May-June 1972. Ionene Polymers. Rembaum A.
Radiat Res. 2003 March; 159(3):371-80. Evaluation of the effect of ascorbic acid treatment on wound healing in mice exposed to different doses of fractionated gamma radiation. Jagetia G C, Rajanikant G K, Rao S K.

1. Compositions comprising one or more vitamins and/or their derivatives in association with an Olea Europea extract, preferably an aqueous extract, and/or one or more of its components, such as oleuropeina, tyrosol and hydroxytyrosol, and/or ionene polymers, along with one or more adjuvants and/or excipients pharmaceutically or cosmetically acceptable.

2. Compositions according to claim 1, wherein said vitamins and/or their derivatives are ascorbic acid and/or its derivatives, and said ascorbic acid is L-ascorbic acid or wherein vitamins are chosen in the group comprising vitamin A, E, D, K, B1, B2, B3, B5, B6, B8, B9, B12.

3-7. (canceled)

8. Compositions according to claim 1, further comprising Echinea purpurea extract, angustifolia or pallida and/or one or more active principles contained in Echinea purpurea extract, angustifolia or pallida, and wherein said one or more active principles contained in Echinea purpurea extract, angustifolia or pallida are chosen from the group comprising heteroxylanes, rhamno-arabino-galactanes, glycoproteines, cichoric acid, alkylamides, caffeic acid derivatives, flavonoids, caryophyllene.

9-11. (canceled)

12. Compositions according claim 1, further comprising one or more compounds chosen from the group comprising polymeric ethers, monohydric alcohols, polyhydric alcohols, tetraborates or thiosulfates.

13-15. (canceled)

16. Compositions according to claim 1, further comprising one or more substances included in the group comprising collagen, fibrin glue, fibrin and its derivatives, dura mater.

17. (canceled)

18. Compositions according to claim 1, further comprising one or more compounds chosen from the group comprising hyaluronic acid or its derivatives, hydroxymethylcellulose, maltodextrines, amino acids.

19. Compositions according claim 1, in the form of serum, gel emulsions, creams, medical devices.

20. (canceled)

21. Compositions according to claim 1, wherein ionene polymers have the general formula (I):
[—N(CH₃)₂—(CH₂)ₓ—N(CH₃)₂—(CH₂)_{y}-].2Z⁻ (I)
wherein x and y are integral numbers and Z is an halide.

22. Compositions according to claim 21, wherein Z is Br or Cl, wherein ionene polymer is obtained by reaction of 1,4-dichlorobutane with tetra-methylen-diamine.

23. (canceled)

24. Compositions according to claim 1, wherein ionene polymers are chosen among those obtained by reaction of 1,4-dichlorobutane with poly(oxyethylene(dimethylimino)-ethylene(dimethylimino)ethylene dihalides), poly(2-hydroxyethylene-dimethyliminio-2-hydroxypropylene-dimethylimino methylene)dihalides, poly[{alkyl-(3-ammoniopropyl)imino}trimethylene dihalides], poly-[dialkyl-imino)ethylene halides] or with poly-[(hydroxy-dialkyl-imino)ethylene halides.

25. Composition according to claim 1, wherein ascorbic acid or its derivatives are present at a concentration between 0.1% and 35%.

26-29. (canceled)

30. Composition according to claim 1, wherein ionene polymers are present at a concentration between 0.001% and 10%.

31. (canceled)

32. Composition according to claim 1, wherein extract of Olea Europea is present at a concentration between 0.1% and 95%.

33-41. (canceled)

42. Compositions according to claim 1, comprising 20% water, preferably bi-distilled water, 20% L-ascorbic acid, 2.66% ionene polymer having formula (I), with x=3 and y=3, 0.3% potassium thiosulfate, 22% aqueous extract of Olea Europea, 15% polyoxyethylene/polyoxypropylene polymer, 20% of a mixture 50:50 of propylenglycol and 1,2 butylenglycol.

43. Compositions according to claim 1, comprising 14.2% L-ascorbic acid, 26.8% aqueous extract of Olea Europea, 44% etoxyglycol, 0.06% citric acid, 14.2% depurated water.

44-45. (canceled)

46. Compositions according to claim 1, comprising vitamins A and E in association with one or more ionene polymers, along with one or more adjuvants and/or excipients, pharmaceutically or cosmetically acceptable.

47-50. (canceled)

51. Compositions according to claim 46, further comprising one or more compounds chosen in the group comprising cogic acid, azelaic acid, fitic acid, glycolic acid, lactic acid, fumaric acid, tartaric acid, L-aspartic acid, L-ascorbic acid, fitic acid, Arbutin, or comprising ionene polymer between 0.01% and 10% in weight, cogic acid between 1% and 10% in weight, azelaic acid between 1% and 30% in weight, glycolic and/or lactic acid between 1.5% and 15% in weight, trans-cis retinic acid between 0.01% and 5% in weight, acetate E vitamin between 0.5% and 5% in weight, hydrating base cream comprising at least an emollient or wetting-agent selected from a group comprising cetyl esters, cetyl alcohol, glycerine, a preservative selected from the group comprising methyl paraben, propyl paraben and laurylsulfate sodium as emulsifying agent, and finally water up to 100% in weight.

52-68. (canceled)

69. Compositions according to claim 1 for use in the medical field, or for use cosmetic treatment.

70-79. (canceled)

80. Use of ionene polymers and/or Olea Europea for stabilising compositions comprising vitamins.

81. (canceled)

82. Use according to claim 80, wherein vitamin are chosen from the group comprising vitamin A, B1, B2, B3, B5, B6, B8, B9, B12, C, D, E, K.
